(19) United States
(12) Patent Application Publication
(10) Pub. No.: US 2003/0069515 A1
(43) Pub. Date: Apr. 10, 2003

(54) Device for detecting the condition of heat of an animal, a positioning device, a stable provided with a positioning device and a method of detecting a conditon of heat

(75) Inventors: Antoon Peter Andre Theelen, Maassluis (NL); Elena Espada Aventin, Delft (NL)

Correspondence Address: Penrose Lucas Albright, MASON, MASON & ALBRIGHT, 2306 South Eads Street, P.O. Box 2246, Arlington, VA, 22202, US

(73) Assignee: Lely enterprises AG, a Swiss Limited Liability Company

(21) Appl. No.: 10/262,080

(22) Filed: Oct. 2, 2002

(30) Foreign Application Priority Data
Oct. 4, 2001 (NL) 1019107

(51) Int. Cl. A61B 10/00

(52) U.S. Cl. 600/551

(57) ABSTRACT

A device for detecting the condition of heat of an animal, in particular a pig, comprising a reaction-detecting means for detecting a heat-related reaction, such as a standing-reflex. There is disposed at least one stimulation means for stimulating the animal to give the heat-related reaction. The demonstrated reaction is compared with a reaction which is characteristic of a particular condition of heat.

The invention relates to a device for detecting the condition of heat of an animal according to the preamble of claim 1.

Such a device is known from Dutch patent NL-C-1006296. Said patent discloses a device which is disposed in the vicinity of the animals to be examined. The device is provided with a frame having the external features of the animal species. Animals which are on heat will demonstrate a heat-related behaviour around the device, such as mounting the frame. The behaviour around the frame can be detected electronically or by means of a camera including picture processing. By means of electronic animal recognition the number of contacts of an individual animal can be registered.

A disadvantage of the above-mentioned device is that the phase of the on-heat-period cannot be observed accurately. This is important, because the phase of the on-heat-period is determinative for the chance of insemination. For example, the so-called insemination phase, i.e. the period within the on-heat-period of for example a pig, during which period insemination can most successfully take place, only covers a small part of the entire period in which heat-related reactions occur. For an accurate determination of the beginning of the insemination phase it is necessary also to determine the phases preceding this insemination phase, i.e. the so-called pre-heat-phase and the first-boar-phase The characteristics of these phases are often not clearly detectable.

The invention has for its object to provide a device which accurately detects the phase of the on-heat-period. A further object is to provide a positioning device being suitable for that purpose and comprising such a device. It is also an object of the invention to provide a suitable stable for such a positioning device. A further object of the invention is to provide a method of detecting a condition of heat of an animal. According to the invention, for the object mentioned first, a device of the above-described type is characterized by the measures of claim 1.

In certain phases of the heat, with pigs the preheat-phase and the first-boar-phase, proper heat-related reactions can only be observed if the oestrous animal is stimulated. These heat-related reactions can be incited by stimulation with the aid of stimulation means.

In a preferred embodiment, the stimulation means can be switched into an active and a non-active position. This makes it possible to distinguish between a heat-related reaction which is given entirely independently by the animal and a reaction which only occurs after active stimulation.

In another embodiment, the stimulation means can be moved so as to come into contact with the animal with the aid of a displacing means. By bringing the stimulation means into contact with the body of the animal, the animal can be stimulated intensively and in this case it is possible also to incite a detectable reaction at a low degree of heat.

In a particular embodiment, the device is provided with position-determining means for determining the position of at least a part of the animal and the displacing means can be controlled with the aid of the data from the position-determining means. Stimulation can take place most effectively on particular places of the animal; often in the vicinity of the genitals. By determining the exact position of the animal, also these places can be positioned accurately, after which the displacing means can position the stimulation means on these places.

In an advantageous embodiment, the stimulation means comprises one or more pressure means for exerting local pressure on the animal. Certain phases in the on-heat-period can only be observed if local pressure is exerted on the animal. This is for example the case with a pig. The pressure is preferably exerted on the flank or on the back of the animal. These places are the most response-sensitive. In a preferred embodiment, the stimulation means can be disposed against the animal under pre-loading. This measure prevents the animal from being injured when moving relative to the pressure means.

In a further embodiment, the reaction-detecting means is integrated in the stimulation means. In this way the reaction-detecting means can be disposed in a simple and cheap manner. This also limits the inconvenience for the animal. It is particularly advantageous when the reaction is detecting means comprises a counter-pressure sensor. In this case it is possible, in a simple manner, to exert pressure on the animal as well as to determine the reaction in the form of a counter-pressure.

In an embodiment, the stimulation means comprises a screen for displaying a heat-inciting, visual picture, in particular a picture of a male animal. It is known that during the on-heat-period female animals react differently on signals from the male animal than in other periods. These signals may consist of smells, sounds or visual pictures.

In a preferred embodiment, the reaction-detecting means comprises one or more movement sensors. Important heat-related reactions consist of moving or not moving in response to a stimulus. These reactions can be detected by means of movement sensors. The movement sensors may comprise one or more light sensors. Light sensors have the advantage of not comprising moving components. The movement sensors may comprise one of more sensors for measuring the weight or a part of the weight of the animal. By measuring the weight it can be detected whether or not an animal is present at a particular place, which is an indication of whether the animal has moved. Besides, the measured weight of the animal provides interesting management information for the operator. The reaction-detecting means may also be a camera. The picture of the entire animal or a part of the animal obtained by the camera can be analysed by means of software and a processing unit. In this manner a picture of an animal in a condition of heat can be distinguished from a picture of an animal in a condition of non-heat.

The second object of the invention is achieved by the measure of claim 15. By combining several functions in one positioning device, these functions can be performed in a compact and cheap manner. There may be provided metering means for supplying feed or cooling means for cooling the animal for the purpose of enticing the animal. By enticing is the animal to go into or through the positioning device, the animal comes voluntarily into contact with the device according to the invention. Coming voluntarily into contact with a device for detecting a condition of heat constitutes part of keeping animals in an animal-friendly manner.

There may be disposed an insemination device for automatically inseminating the animal. The insemination and detection of a condition of heat take place in the same group of animals. Sometimes, for having the greatest chance of impregnation, the animal should be inseminated immediately after the detection. This can already be done when the animal is still present in the positioning device.

There may be disposed selection means for selectively opening or closing an exit by means of detection of a heat-related reaction. With the aid of said means, when the detection of a condition of heat has taken place, the animal can immediately be guided to a suitable accommodation for animals, for example a separation area, for being inseminated, or to an accommodation for pregnant animals in the case that pregnancy of the animal has been established.

A drive means may be disposed for driving the animal to an exit. Hereby it is possible to prevent unwilling animals from occupying the positioning device too long. In a particular embodiment, the stimulation means comprises the drive means. In this manner there can be obtained a simple construction having both a stimulation function and a drive function. In a further embodiment, the drive means can be moved in the direction of the exit. In this manner the animal can be accompanied by unpleasant stimuli during leaving the positioning device.

The third object mentioned is achieved by the measure of claim 25. In a preferred embodiment, in the stable in the vicinity of the positioning device there is provided an accommodation for housing a male animal, a connection for passing on visual signals and/or sounds and/or smells and/or touch signals from the male animal being provided between the positioning device and the accommodation. This enables to generate, in a natural manner, stimuli via the presence of the male animal.

In another embodiment, the stable comprises several accommodations which are accessible to the animal from the positioning device. This makes it possible to guide an animal, if said animal is suitable for being impregnated, directly from the positioning device to an insemination area. Insemination can then take place in said area in an artificial manner by an inseminator or in a natural manner by a male animal.

The fourth object mentioned is achieved by the measure of claim 29. In a first step, stimulation of the animal takes place to induce a heat-related reaction. Then a detection of a heat-related reaction, if any, takes place. The reaction is subsequently compared with a reaction which is characteristic of a particular condition of heat. By storing several reactions which are characteristic of a condition of heat in a memory and comparing the demonstrated reaction herewith, the on-heat-phase can be established.

In a preferred embodiment, identification of the animal takes place. This makes it possible to register which animal is momentarily treated by the device and when this has been done in the past.

In a preferred embodiment, the stimulation takes place with the aid of heat-related data of the animal stored in the memory of the detecting device. Thus it is prevented that too much or too little stimulation of an animal takes place, which would result in unrest and unnecessary occupation of the measurement area. In this manner, the age, the fertility status or the sensitivity of the animal to stimulation can be taken into account.

In an embodiment of the method, the stimulation takes place on a specific place of the animal and said specific place is determined with the aid of position-determining means. In this manner it is possible to stimulate the animal on those places that demonstrate the most obvious reaction.

In another preferred embodiment of the method, the stimulation takes place by exerting local pressure on the animal. Inter alia with pigs this is a known stimulus for demonstrating a heat-related reaction. The pressure is then exerted in particular on the flank and/or the back of the pig.

In a preferred embodiment, the intensity of the stimulation is increased during the stimulation. By means of this method there is a smaller risk of a startling reaction of the animal when the latter is stimulated.

In another preferred embodiment of the method, the heat-related reaction implies moving or not moving out of the measurement area. Inter alia with pigs it is known that moving or not moving after stimulation makes the difference between two phases in the on-heat-period.

In an advantageous embodiment of the method, there is generated a signal for a follow-up reaction after a particular condition of heat has been established. This makes it possible immediately to link a follow-up action with the establishment of said particular condition of heat.

In a preferred embodiment of the method, by the signal a drive means is activated to drive the animal from the measurement area. In a particular embodiment, the drive means is only activated if it is established that the animal leaves the measurement area insufficiently quickly. Hereby it is prevented that unpleasant stimuli are given unnecessarily to an animal.

In another preferred embodiment of the method, an automatic insemination device is activated by the signal. The ideal impregnation period is often of short duration. By automatically inseminating immediately after detection of said ideal impregnation period, the greatest possible chance of a successful impregnation is obtained. The impregnation may also take place in a natural manner via the male animal or in a known manual manner by an inseminator. In these last mentioned cases an entrance for the animal to an insemination area is released by the signal. Immediately after detection the female animal is guided to a separate insemination area where impregnation takes place by the male animal or by the inseminator.

In a preferred embodiment of the method, a specific sperm sample for insemination of the animal is released by the signal from a stock of sperm samples. In this manner it is possible to input beforehand into a control unit with which sperm the specific animal has to be inseminated when it enters the fertility period. This specific sperm is only released for insemination if the specific animal is ready for insemination. This can be done both in the case of automatic and manual insemination. Especially with manual insemination the error of inseminating an animal with the wrong sperm can easily be made. In practice the inseminations are often performed by several persons who have to make the correct choice from several doses. This can easily lead to errors.

In an embodiment of the method, the signal comprises a warning signal for an operator. This may be for example an SMS-signal on the mobile telephone of said operator or a signal to a generator of an attention list. The operator's attention is hereby drawn to the fact that the relevant animal has to be watched closely or has to be separated for insemination.

The invention will be set out in further detail hereinafter with reference to figures of exemplary embodiments, in which:

FIG. 1 is a side view of a positioning device according to the invention;

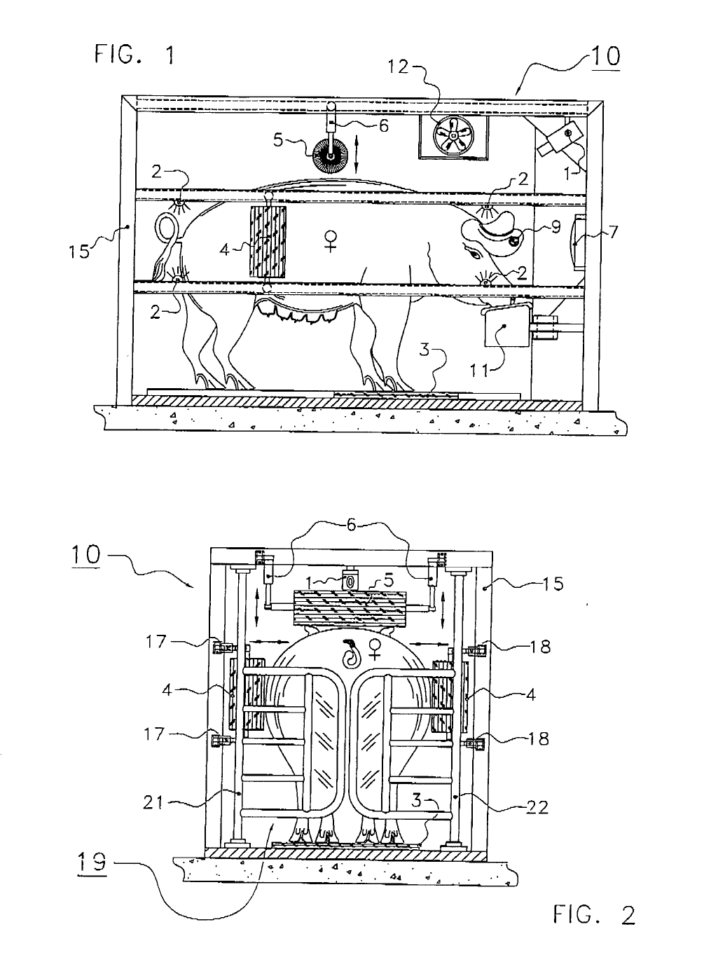

FIG. 2 is a rear view of the positioning device of FIG. 1;

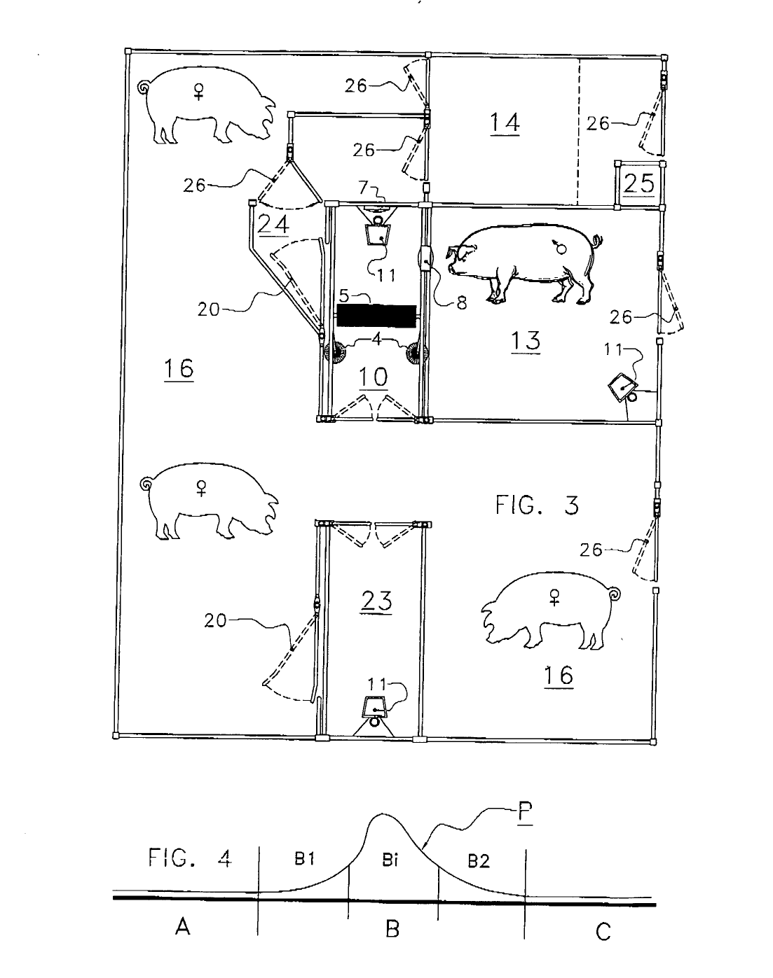

FIG. 3 is a schematic plan view of a stable according to the invention, and

FIG. 4 is a graph in which the on-heat-periods of a pig are indicated.

FIG. 1 shows in a side view a positioning device 10 according to the invention in the form of a feeding device for pigs. Said positioning device 10 is in particular suitable for groups of sows. The device comprises a frame 15 for separating the area where the animal is present and to screen it off from the other areas. Due to this, an animal which is present in the positioning device 10 is not bothered by other animals in the stable. Apart from this, the frame 15 serves to fasten auxiliary means to it. There is provided a feed trough 11 for automatically and individually supplying feed via a metering device. For this purpose the pig wears in one of its ears a transponder 9. An identification device recognises the pig by means of said transponder and the controlling computer can supply the desired amount of feed. A weighing device 3 measures the weight of a front part of the pig. By means of comparison data the measured weight can be reduced to the total weight. Movements of the animal lead to fluctuations in the weight. In this manner the weighing device can be used for detecting whether the animal stands still. There are disposed light sensors 2 for determining the position of the animal in the positioning device 10. These light sensors are also used for indicating a change of position. The camera 1 has the same function. This camera cooperates with the controlling computer for reducing the position and the movement of the animal from the recorded picture. There is disposed a display screen 7 in the animal's field of vision. On said screen there are shown pictures of a boar, which pictures incite a reaction with the animal. The pictures are preferably motion pictures and provided with corresponding sounds. On the inside of the two lateral sides of the frame 15 and on its upper side there are disposed pressure means 4, 5 for exerting local pressure on the animal, respectively in the flanks and on the back. These pressure means 4, 5 are designed as rolls, so that the animal is allowed to move freely to a certain extent in the longitudinal direction. By means of cylinders 6, 17, 18 each pressure means 4, 5 can be moved towards the animal. The cylinders 6, 17, 18 are controlled by the controlling computer. Before the cylinders 6, 17, 18 are controlled, first the exact position of the animal is determined. This takes place with the aid of data from the camera 1 and the light sensors 2. The entrance and the exit of the positioning device 10 are designed to be closed by gates 19, 20 (FIG. 3). FIG. 2 clearly shows the entrance. The gates can be operated by the controlling computer. The entrance gate 19 consists of two gate portions 21, 22, which are each hingeably fastened to the frame 15.

FIG. 3 shows the positioning device 10 in a stable. A group of sows is present in the sow accommodation 16. The sow accommodation 16 is provided with a feeding station 23. The positioning device 10 according to the invention is centrally disposed in the sow accommodation 16. Next to said device 10 there is provided a boar accommodation 13 for housing a seeking boar. Via a sniff-opening 8 between the positioning device 10 and the boar accommodation 13, contact between the animal present in the positioning device 10 and the seeking boar can take place. When contact is not desired, the sniff-opening 8 can be closed. The exit of the positioning device 10 is connected with a selection device 24 having two exits. One exit leads to an insemination area 14, the other to the sow accommodation 16. In the insemination area 14 there may be present a boar for natural insemination, but said area is preferably accessible to an inseminator for artificial insemination. There is provided a releasing device 25 for releasing a specific dose for the inseminator from a collection of sperm doses. For this purpose, the choice of the relevant dose has to be inputted beforehand into the controlling computer.

With reference to FIG. 4 the different phases in the on-heat-period of the sow can be elucidated. There are distinguished three main phases in time, indicated by A, B and C. The pre-heat-phase is indicated by A. When a sow is in this phase, there only occurs a standing-reflex at pressure in the flanks of the animal. The standing-reflex is a reaction in response to a stimulus at which the sow hardly moves. In the real on-heat-phase B there can be distinguished three sub-periods. In the so-called first-boar-phase B1 the sow demonstrates a standing-reflex at a pressure in the flanks, as well as after pressure on the back and after having seen a boar. The insemination phase Bi can be distinguished from the phase B1 in that the standing-reflex is also demonstrated after having seen a person. In the second-boar-phase B2 the sow reacts in the same manner by a standing-reflex as in the first-boar-phase. In the post-heat-phase C the sow demonstrates normal behaviour and does not react to a stimulus by a standing-reflex. The line indicated by P shows the chance of a successful impregnation after insemination. It is visible that insemination has to take place in the insemination phase Bi, and in particular in the first ¾ part thereof. Therefore, exact determination of the phase in which the sow is at that moment, is important for taking the decision at what point of time the sow has to be inseminated.

The device operates as follows. A sow presents herself to enter the positioning device 10. The sow is attracted for example by the feed which is supplied therein or because she wishes to be pampered for example by the cooling device 12. The animal is identified by the identification device via a transponder 9, preferably disposed in the ear of the animal. With the aid of data in the memory of the controlling computer it is decided whether the sow has to be stimulated. This needs not to take place with sows whose condition of heat is stable, for example with sows which are certainly pregnant or with sows which have had a fertility period a short time ago. In order not to charge the device unnecessarily, these animals are not admitted. For animals whose condition of heat is not clear, the entrance gate 19 is opened. After an animal has entered, the gate 19 is re-closed to prevent disturbance by other sows. The on-heat-phase is detected with the aid of the demonstration of a standing-reflex after a stimulus. With a sow which has not yet demonstrated a standing-reflex, there is exerted pressure on the flank by shifting the side-pressure-rolls 4 towards the centre of the device 10 until a particular counter-pressure has been attained. Due to the tact that the contact point of the pressure means 4, 5 is situated on a roll, the animal is allowed to move forwards or rearwards to a certain extent without being injured. Subsequently there is applied a stimulus for moving in the form of the opening of the gate 20 or the supply of a portion of feed to the feed trough 11. Movement can be registered by a camera 1, by a change of weight on the weighing plate 3, by the movement sensors in the pressure rolls 4, 5 or by light sensors 2. If the animal demonstrates less movements than a particular minimum, the so-called movement limit, then it is assumed that the animal demonstrates a standing-reflex. The animal is then in phase A or B. Subsequently there is applied a lower stimulus and the higher stimulus is removed. For this purpose the back-pressure-roll 5 is lowered until a particular counter-pressure has been attained. Subsequently the side-pressure-rolls 4 are removed. If the animal continues to demonstrate the standing-reflex, it is obvious that the animal is in phase B and has to be inseminated within one day. This can be automatically signalled to the operator. If the animal demonstrates movement, then the animal is in phase A and has to be inseminated within 1 to 2 days. Then the animal has to be examined again on heat at any rate within one day. As an additional check a video-motion-picture of a boar with corresponding sound can be shown to the animal on the screen 7. Then the back-pressure-roll 5 is removed. Also in response to this stimulus an animal in phase B will demonstrate a standing-reflex. Instead of showing a video picture of a boar, the shutter before the sniff-opening 8 can be opened, so that the animal can see a seeking boar present in the boar accommodation 13. A stimulus by seeing and/or smelling and/or feeling a seeking boar has more effect than a stimulus by means of a video picture. If the video picture of the boar is replaced by a picture of a person and the animal continues to demonstrate a standing-reflex, it is in the insemination phase (Bi). The animal is immediately guided to the insemination area 14. To this end, the controlling computer controls the gate 20 to be opened and controls the selection device 24. After having been inseminated, the sow can come back via the gate 26 into the sow accommodation 16. In order to see whether the inseminated sow is pregnant, the sow will be guided again through the device 10 in the next cycle. If no standing-reflex is detected then, the controlling computer will consider the relevant sow as definitively pregnant. Until throwing has taken place, the sow will not be admitted to the device 10.

The controlling computer can determine with the aid of the signals from the light sensors 2, the weighing floor 3, the camera 1 or the movement sensors on the pressure rolls 4, 5, whether and where the animal is located in the positioning device 10. If it is detected that the animal is still present in the positioning device 10 at a moment when a certain time has elapsed after the opening of the gate 20, then an unpleasant stimulus is given to the animal by a drive means in order to drive the animal to an exit. For that purpose the stimulation means can be used. This may be done by giving an electric shock via an electric contact point disposed on one of the pressure means 4, 5. Unpleasant stimuli may also be given in the form of sound produced by a loudspeaker, or in the form of a frightening picture by the screen 7. Driving is also possible by pressing the pressure roll 4, 5 with an unpleasantly high pressure against the animal. By disposing the stimulation means, preferably the back-pressure-roll 5, in such a way that it is movable in the direction of the exit, the animal can be accompanied by unpleasant stimuli during leaving the positioning device 10.

If no standing-reflex of an animal present in the device is established, whereas said animal has demonstrated a standing-reflex whilst passing through the device in the past days, the animal is probably in phase C. Insemination is no longer useful and the next fertility cycle, which starts after about 19 days, will have to be waited for. Until that time the controlling computer blocks the access to the device for the relevant animal.

The feed trough 11 is not essential to the device 10. The feed trough 11 with the metering device may also be disposed beyond the exit so as to cause less distraction in the device 10 and yet to act as enticing means, so that the sows like to pass through the device 10.

1. A device for detecting the condition of heat of an animal, in particular a pig, comprising a reaction-detecting means (1, 2, 3) for detecting a heat-related reaction, such as a standing-reflex, characterized in that there is disposed at least one stimulation means (4, 5, 7) for stimulating the animal to give the heat-related reaction.

2. A device as claimed in claim 1, characterized in that the stimulation means (4, 5) can be switched into an active and a non-active position.

3. A device as claimed in claim 1 or 2, characterized in that the stimulation means (4, 5) can be moved so as to come into contact with the animal with the aid of a displacing means (6).

4. A device as claimed in claim 3, characterized in that the device is provided with position-determining means (1, 2) for determining the position of at least a part of the animal, and in that the displacing means (6) can be controlled with the aid of the data from the position-determining means (1, 2).

5. A device as claimed in any one of the preceding claims, characterized in that the stimulation means comprises one or more pressure means (4, 5) for exerting local pressure on the animal.

6. A device as claimed in any one of claims 3 to 5, characterized in that the stimulation means (4, 5) can be disposed against the animal under pre-loading.

7. A device as claimed in any one of the preceding claims, characterized in that the reaction-detecting means is integrated in the stimulation means (4, 5).

8. A device as claimed in any one of the preceding claims, characterized in that that the reaction-detecting means comprises a counter-pressure sensor.

9. A device as claimed in any one of the preceding claims, characterized in that the stimulation means comprises a screen (7) for displaying a heat-inciting visual picture, in particular a picture of a male animal.

10. A device as claimed in any one of the preceding claims, characterized in that there are disposed identification means (9) for identifying the animal.

11. A device as claimed in any one of the preceding claims, characterized in that the reaction-detecting means comprises one or more movement sensors.

12. A device as claimed in claim 11, characterized in that the movement sensors comprise one or more light sensors (2).

13. A device as claimed in claim 11 or 12, characterized in that the movement sensors comprise one or more weight sensors (3) for measuring the weight or a part of the weight of the animal.

14. A device as claimed in any one of claims 1 to 11, characterized in that the reaction-detecting means comprises a camera (1).

15. A positioning device (10) provided with means (15) for keeping an animal in an at least substantially defined position, provided with means for an animal-related treatment and provided with a device as claimed in any one of the preceding claims.

16. A positioning device (10) as claimed in claim 15, characterized in that there are disposed metering means (11) for supplying fodder or water.

17. A positioning device (10) as claimed in claim 15 or 16, characterized in that there are disposed cooling means (12) for cooling the animal.

18. A positioning device (10) as claimed in any one of claims 15 to 17, characterized in that there is disposed an insemination device for automatically inseminating the animal.

19. A positioning device (10) as claimed in any one of claims 15 to 18, characterized in that there is disposed a milking robot for milking the animal.

20. A positioning device (10) as claimed in any one of claims 15 to 19, having an entrance and at least one exit, characterized in that the entrance is designed to be closed.

21. A positioning device (10) as claimed in any one of claims 15 to 20, having several exits, characterized in that there are disposed selection means for selectively opening or closing an exit with the aid of detection of a heat-related reaction.

22. A positioning device (10) as claimed in any one of claims 15 to 21, characterized in that a drive means is disposed for driving the animal to an exit.

23. A positioning device (10) as claimed in claim 22, characterized in that the stimulation means (4, 5, 7) comprises the drive means.

24. A positioning device (10) as claimed in claim 22 or 23, characterized in that the drive means can be moved in the direction of the exit.

25. A stable provided with a positioning device (10) as claimed in any one of claims 15 to 24.

26. A stable as claimed in claim 25, characterized in that in the vicinity of the positioning device (10) there is provided an accommodation (13) for housing a male animal, a connection (8) for passing on visual signals and/or sounds and/or smells and/or touch signals from the male animal being provided between the positioning device (10) and the accommodation (13).

27. A stable as claimed in claim 25 or 26, characterized in that the stable comprises several animal accommodations which are accessible to the animal from the positioning device (10).

28. A stable as claimed in claim 27, characterized in that the accommodations (14) for animals comprise an accommodation for a male animal.

29. A method of detecting a condition of heat of an animal in a measurement area with a device as claimed in any one of claims 1 to 14, characterized in that successively at least the following steps are taken:
stimulation of the animal,
detection of a reaction of the animal,
comparison of the reaction with a reaction which is characteristic of a particular condition of heat for determining the particular condition of heat.

30. A method of detecting a condition of heat of an animal as claimed in claim 29, characterized it that identification of the animal takes place.

31. A method of detecting a condition of heat of an animal as claimed in claim 30, characterized in that the stimulation takes place with the aid of heat-related data of the animal stored in the memory of the detecting device.

32. A method of detecting a condition of heat of an animal as claimed in any one of claims 29 to 31, characterized in that the stimulation takes place on a specific place of the animal, and in that said specific place is determined with the aid of position-determining means (1, 2, 3).

33. A method of detecting a condition of heat of an animal as claimed in any one of claims 29 to 32, characterized in that the stimulation takes place by exerting local pressure on the animal.

34. A method of detecting a condition of heat of an animal as claimed in any one of claims 29 to 33, characterized in that the intensity of the stimulation is increased during the stimulation.

35. A method as claimed in any one of claims 29 to 34, characterized in that the heat-related reaction implies moving or not moving out of the measurement area.

36. A method as claimed in claim 35, characterized in that moving out of the measurement area is determined by means of light sensors (2).

37. A method as claimed in claim 35, characterized in that moving out of the measurement area is determined by changing a value of a weight sensor (3).

38. A method as claimed in any one of claims 29 to 37, characterized in that there is generated a signal for a follow-up action after a particular condition of heat has been established.

39. A method as claimed in claim 38, characterized in that by the signal a drive means is activated to drive the animal from the measurement area.

40. A method as claimed in claim 39, characterized in that the drive means is only activated if it is established that the animal leaves the measurement area insufficiently quickly.

41. A method as claimed as claimed in any one of claims 38 to 40, characterized in that an automatic insemination device is activated by the signal.

42. A method of detecting a condition of heat of an animal as claimed in any one of claims 38 to 41, characterized in that an entrance for the animal to an insemination area (14) is released by the signal.

43. A method of detecting a condition of heat of an animal as claimed in any one of claims 38 to 42, characterized in that a specific sperm sample for insemination of the animal is released by the signal from a stock of sperm samples.

44. A method of detecting a condition of heat of an animal as claimed in any one of claims 38 to 43, characterized in that the signal comprises a warning signal for an operator.
